(19) United States
(12) Patent Application Publication
(10) Pub. No.: US 2013/0323119 A1
(43) Pub. Date: Dec. 5, 2013

(54) SYSTEM AND METHOD FOR DISINFECTION OF MEDICAL DEVICES

(71) Applicant: Jim Alwan, Ramona, CA (US)

(72) Inventor: Jim Alwan, Ramona, CA (US)

(73) Assignee: CareFusion 303, Inc., San Diego, CA (US)

(21) Appl. No.: 13/486,879

(22) Filed: Jun. 1, 2012

(51) Int. Cl. A61L 2/10

(52) U.S. Cl. 422/24; 250/455.11

(57) ABSTRACT

An apparatus configured to disinfect an intravenous (IV) access port is disclosed. The apparatus has at least one sterilizing cap with a body configured to removably couple to the access port and an illuminator coupled to the body. The body and illuminator are configured to expose at least one surface of the access port to a dose of ultraviolet (UV) light. In certain embodiments, the illuminator includes a source of UV light, such as a light-emitting diode (LED). In certain embodiments, the illuminator receives the UV light from a remote source through a fiber-optic cable.

### CROSS-REFERENCE TO RELATED APPLICATIONS

Not applicable.

### BACKGROUND

1. Field

The present disclosure is related to systems and method of disinfecting devices and, in particular, disinfecting medical devices using ultraviolet (UV) radiation.

2. Description of the Related Art

Patients in hospitals are often provided with medical fluids that are administered through an intravenous (IV) infusion using assemblies of tubes and fittings commonly referred to as “IV sets.” FIG. 1 illustrates a patient 10 receiving a medical fluid from container 14 through an IV set 18 using an infusion system 12 that includes a control module 16 and a pumping module 20. A patient receiving certain medications or with other needs, such as total parenteral nutrition in a chronically ill patient, may have a central venous catheter (CVC) installed. FIG. 2 depicts a patient 10 with a CVC 30 having a lumen 32 that is inserted into the jugular vein. This particular CVC 30 has three access ports 34 to allow simultaneous delivery of multiple medical fluids or extraction of a blood sample without the need to disconnect an IV line. A CVC may remain in place for days or weeks, in the absence of problems such as clotting or infection.

Blood Stream Infections (BSIs) are a dangerous, costly, and persistent problem in healthcare, particularly for long-duration devices that penetrate the skin, such as a CVC. One identified cause of BSI is the contamination of IV access ports leading to intraluminal colonization and infection that may lead to sepsis and death. Maintaining sterile access ports is a challenge since these devices are proximal to patient skin, frequently handled by healthcare workers, and may be touched by visitors or come in contact with contaminated surfaces in the hospital. Episodic cleaning of the external surfaces of access ports immediately prior to connection of an IV line using wipes or cleaning solutions is subjective and it is difficult to maintain high levels of compliance. Furthermore, those solutions may contain materials that interfere with valve operation.

### SUMMARY

It is desirable to provide a system and method of sterilizing portions of medical devices that may provide an entrance point for microorganisms to enter a patient's bloodstream, particularly IV access ports, without requiring a technique-dependent cleaning activity by the user.

In certain embodiments, an apparatus configured to disinfect an IV access port is disclosed. The apparatus includes at least one sterilizing cap comprising a body configured to removably couple to the access port, and an illuminator coupled to the body. The body and illuminator are configured to expose at least one surface of the access port to a dose of UV light.

In certain embodiments, a method of disinfecting an IV access port is disclosed. The method includes the step of coupling a sterilizing cap to the access port, wherein the sterilizing cap is coupled to a source of UV light and is configured to expose at least one surface of the access port to a dose of the UV light. The method also includes the step of enabling or actuating the UV light source automatically upon completion of the coupling of the sterilizing cap to the access port.

### BRIEF DESCRIPTION OF THE DRAWINGS

The accompanying drawings, which are included to provide further understanding and are incorporated in and constitute a part of this specification, illustrate disclosed embodiments and together with the description serve to explain the principles of the disclosed embodiments. In the drawings:

FIG. 1 depicts a patient receiving a medical fluid through an IV infusion using an infusion pump.

FIG. 2 depicts a patient with a central venous port inserted into the jugular vein.

FIG. 3 is a plot of MPEs calculated according to the IEC-60825-1 for certain wavelengths of light vs. exposure time.

FIG. 4A depicts an exemplary embodiment of a sterilization system according to certain aspects of the present disclosure.

FIGS. 4B-4D are cross-sections of embodiments of the sterilizing cap according to certain aspects of the present disclosure.

FIGS. 5A and 5B depict additional embodiments of a sterilization system according to certain aspects of the present disclosure.

FIG. 6 depicts another embodiment of a sterilization system integrated into an infusion pump according to certain aspects of the present disclosure.

FIG. 7 depicts a detail of multiple sterilizing caps of a sterilization system coupled to multiple access ports of a CVC according to certain aspects of the present disclosure.

FIG. 8A and 8B depict a sterilizing system that provides mechanical cleaning and UV sterilization according to certain aspects of the present disclosure.

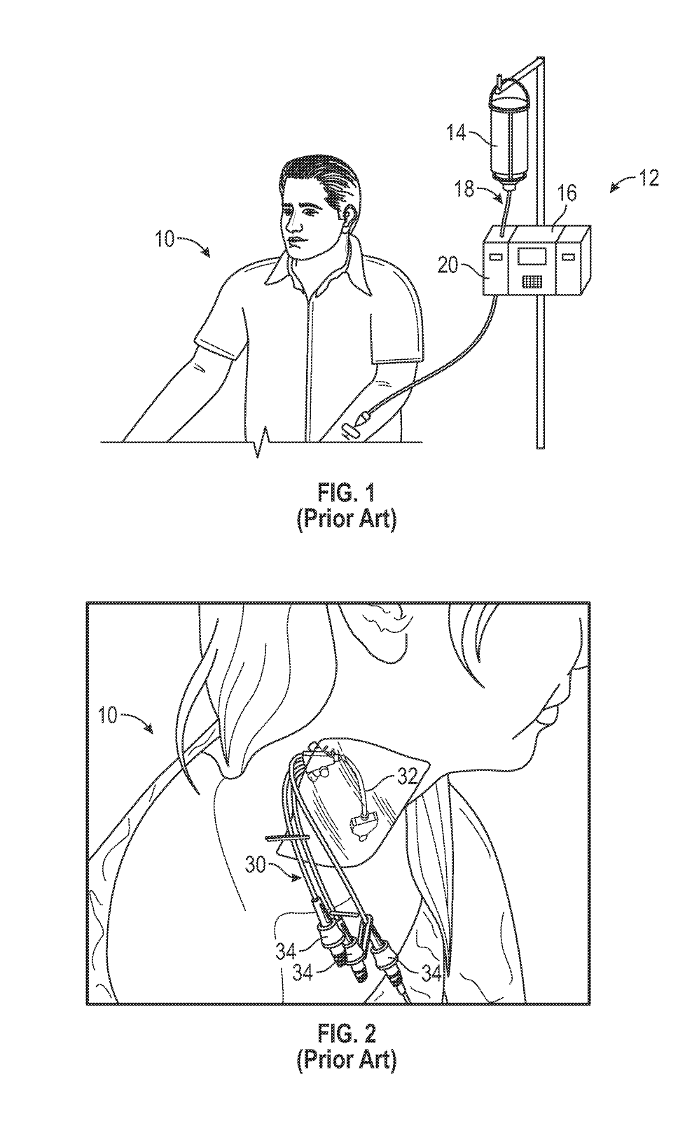

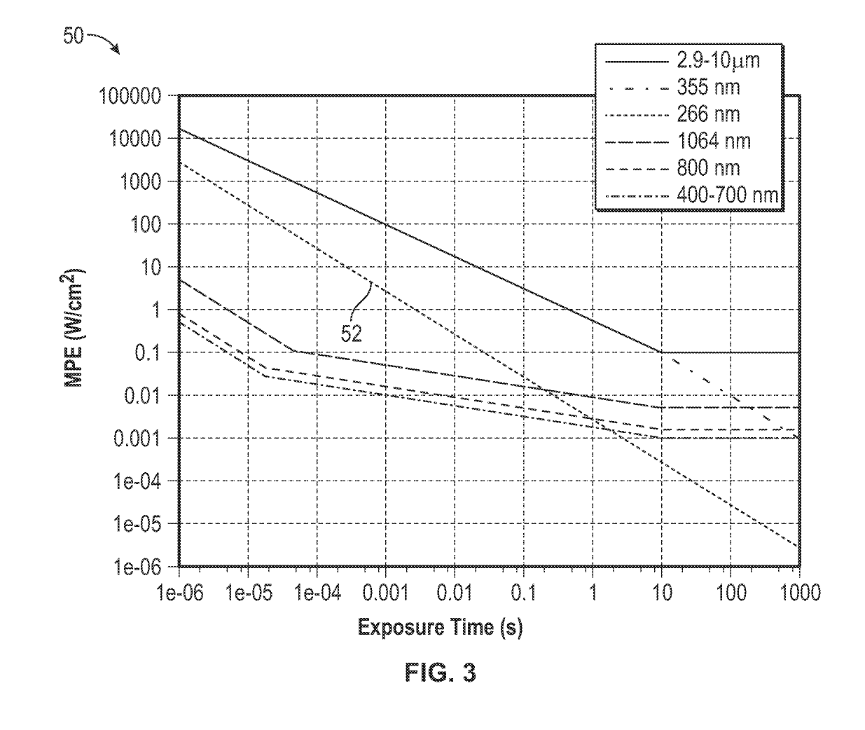

### DETAILED DESCRIPTION

The following description discloses embodiments of a sterilizing system and method suitable for sterilizing internal and external surfaces of medical equipment. In certain embodiments, the system is configured to sterilize an access port of an IV set. In certain embodiments, the system includes a main module coupled to a sterilizing connector via a cable. In certain embodiments, the system includes a small module suitable for direct connection to an access port. It will be recognized that access ports are typical of needleless connectors, and descriptions of use of the disclosed system with access ports is considered to cover use with any tube of fluid connector, including male and female needleless connectors, male and female luer connectors, fittings on syringes and other fluid devices, and all other types of connectors used with fluid handling equipment and treatments.

The detailed description set forth below is intended as a description of various configurations of the subject technology and is not intended to represent the only configurations in which the subject technology may be practiced. The appended drawings are incorporated herein and constitute a part of the detailed description. The detailed description includes specific details for the purpose of providing a thorough understanding of the subject technology. However, it will be apparent to those skilled in the art that the subject technology may be practiced without these specific details. In some instances, well-known structures and components are shown in block diagram form in order to avoid obscuring the concepts of the subject technology. Like components are labeled with identical element numbers for ease of understanding.

As used within this disclosure, the terms “optical” and “light” refer to electromagnetic radiation from ultraviolet to infrared, including wavelengths in the range of 10 nanometers (nm) to 1 millimeter (mm) and includes, but is not limited to, light visible to the human eye, which covers the approximate range of 380-760 nm.

As used within this disclosure, the terms “ultraviolet light” and “UV light” refer to light having a wavelength in the approximate range of 10-400 nm.

As used within this disclosure, the term “ultraviolet A,” abbreviated as “UVA,” refers to light having a wavelength in the range of approximately 315-400 nm.

As used within this disclosure, the term “ultraviolet B,” abbreviated as “UVB,” refers to light having a wavelength in the range of 280-315 nm.

As used within this disclosure, the term “ultraviolet C,” abbreviated as “UVC,” refers to light having a wavelength in the range of 100-280 nm.

As used within this disclosure, the terms “sterilized” and “disinfected” mean that the population of DNA-based microorganisms on a surface has been reduced compared to the population present on the same surface prior to the sterilizing or disinfecting treatment. The amount of reduction for a particular application, for example IV products, may be selected by the hospital or industry organization as to achieve sufficient reduction for the application, for example a reduction in BSIs. As the magnitude of the reduction asymptotically approaches but never reaches zero, there will always be some microorganisms present on the sterilized or disinfected surface no matter what duration of treatment is chosen.

As used within this disclosure, the term “eye safe” refers to an intensity of optical radiation that is generally considered to be safe for long-term exposure to the human eye.

Exposure to UVC light kills microorganisms that utilize deoxyribonucleic acid (DNA) at a rate that asymptotically approaches 100% over time. It has been reported (for example, by Von Sonntag, 1986, Disinfection of free radicals and UV-radiation. International Workshop on Water Disinfection, Compagnie Generale des Eaux, Mulhouse) that DNA has absorption peaks at 200 nm, where the DNA absorbs energy in the ‘backbone’ molecules of ribose and phosphate, and at 265 nm, where the DNA absorbs energy mainly in the nucleobases: cytosine, guanine, adenine, and thymine. The absorbed energy breaks the molecular bonds of certain molecules and causes the bonding of other molecules, such as the formation of thymine dimers when two adjacent thymine molecules become fused. This damage prevents the DNA from being able to replicate, effectively killing the cell. UV light can also damage RNA as well as cell proteins and enzymes that further inhibit growth and function of the microorganism.

Table 1 lists representative dosages required to achieve various “kill rates” of certain common organisms when exposed to UV light. The actual dosage depends on the energy distribution of the light emitted by a particular source within the UV band and the particular sensitivity of each organism to various wavelengths of UV light. For example, a source that emits most of its light at 265 nm may be more effective against particular microorganism than another source that emits most of its light at 400 nm.

TABLE 1

| | kill rate | | |
|---|---|---|---|
| Pathogen | 90% | 99% | 99.99% |
| Legionella pneumophila | 2.0 | 4.0 | 8.0 |
| Staphylococcus aureus | 2.6 | 5.2 | 11 |
| Listeria monocytogenes | 3.4 | 6.8 | 14 |
| Pseudomonas aeruginosa | 5.5 | 11 | 22 |
| Salmonella enteritidis | 7.6 | 16 | 31 |
| Bacillus subtilis (spores) | 12 | 24 | 48 |

Dose (given in mW-sec/cm²) to achieve the stated kill rate

A “dose” is the product of an intensity of the radiation (in units of energy per unit area) multiplied by the duration of exposure (in units of time). For example, the 99.99% kill rate for Listeria monocytogenes, 14 milliwatt-seconds per square centimeter (mW-sec/cm²), can be achieved by exposing a surface to UV light at an intensity of 1 mW/cm² for 14 seconds, or 2 mW/cm² for 7 seconds, or 14 mW/cm² for 1 second.

One generally accepted definition for “sterile” in the medical industry is a “one in a million” probability of a viable microorganism remaining on a surface, which is equivalent to a 99.9999% kill rate. This kill rate will require exposure durations of approximately six times the duration shown for a 90% reduction at the same intensity. The system and method disclosed herein are not limited to a particular level of sterilization, however, and the scope of the present application is limited only by the terms of the appended claims.

Light-emitting diodes (LEDs) that emit light primarily at wavelengths in the UV band are now becoming available at power levels of 1-10 mW. As with previous types of LEDs, it can be expected that the power levels of available UV LEDs will rise while the prices fall as time goes by.

Radiation of any frequency becomes hazardous at some level of exposure. For light, the maximum permissible exposure (MPE) is the highest power or energy density (in W/cm² or J/cm²) of a light source that is considered safe, i.e. that has a negligible probability for creating damage to a human eye in the worst-case scenario in which the eye lens focuses the light into the smallest possible spot size on the retina for the particular wavelength and the pupil is fully open. The IEC-60825-1 and ANSI Z136.1 standards include methods of calculating MPEs.

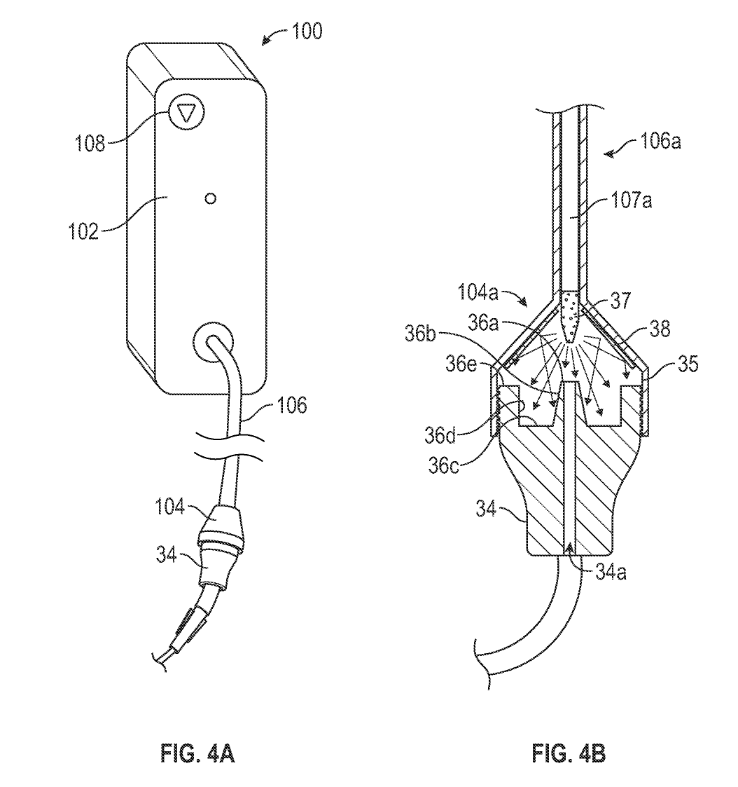

FIG. 3 is a plot 50 of MPEs calculated according to the IEC-60825-1 for certain wavelengths of light vs. exposure time. For example, for light having a wavelength of 266 nm, the line 52 indicates the intensity of the MPE for exposures in the range of 1 microsecond (plotted as 1e-06 seconds) to 1000 seconds. Both the intensity on the vertical axis and the time plotted on the horizontal axis are plotted on a logarithmic scale. Exposure to light having a wavelength of 266 nm at an intensity of 1 mW/cm² (plotted as 0.001 W/cm² in the plot of FIG. 3) is considered safe for an exposure of less than approximately 3 seconds, while light having an intensity of 0.1 mW/cm² is safe for an exposure of less than approximately 30 seconds. It can be seen that different wavelengths follow different curve shapes, due in part to the absorption characteristics of the human eye.

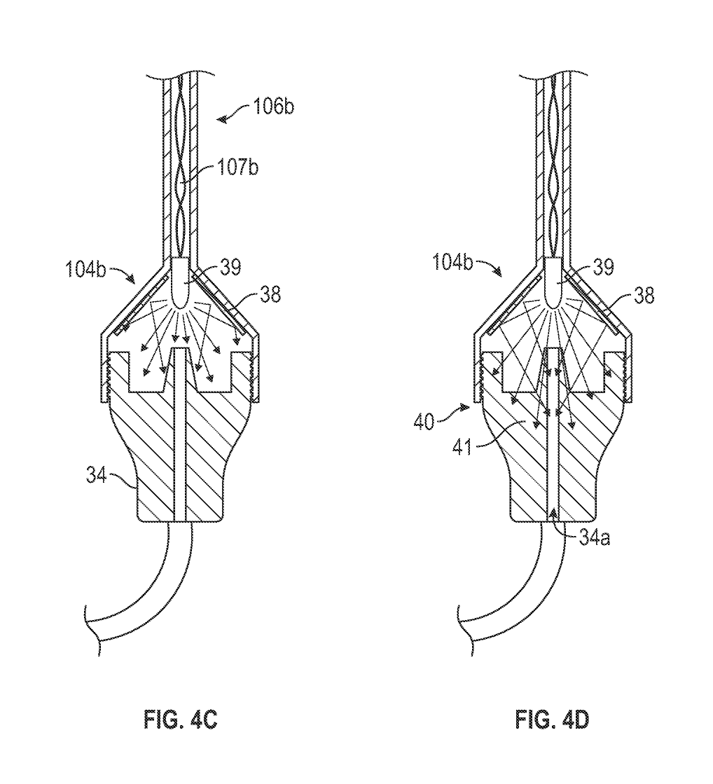

FIG. 4A depicts an exemplary embodiment of a sterilization system 100 according to certain aspects of the present disclosure. The system 100 includes a source 102 connected via a cable 106 to a sterilizing cap 104 that is configured to couple to an access port 34. In general, sterilizing cap 104 is configured to expose certain surfaces of the access port 34 to UV light, as shown in greater detail in FIGS. 4B and 4C. In certain embodiments, the source 102 generates UV light internally and the cable 106 comprises optical fibers or other light-guiding elements that convey UV light from the source 102 to the sterilization cap 104, which is configured to further guide the UV light from the cable 106 onto the desired surfaces of the access port 34 as shown in FIG. 4B. In certain embodiments, the UV light source within the sterilizing cap 104 comprises a Light Emitting Diode (LED) as shown in FIG. 4C and power is provided by the source 102.

In certain embodiments, the access port 34 comprises a UV-transmissive material such that UV light reaches at least one of the interior surfaces of the access port 34. This allows internal features that might otherwise serve as suitable breeding locations for microorganisms to be disinfected. In certain embodiments, the sterilizing cap 104 is configured to direct the UV light into the certain portions of the access port 34 so as to preferentially illuminate one or more interior surfaces.

FIGS. 4B-4D are cross-sections of embodiments of the sterilizing cap 104 according to certain aspects of the present disclosure. FIG. 4B depicts the internal details of one embodiment of a sterilization cap 104a according to certain aspects of the present disclosure. In this embodiment, the sterilization cap 104 has a body 35 and an illuminator that comprises a transmissive element 37 that receives UV light through optical fibers 107a within a fiber-optic cable 106a from a source 102 and directs the UV light onto various surfaces of the access port 34 such as a tip 36a, a conical luer surface 36b, a flat 36c, an engagement surface 36d, and an end 36e. It can be seen that the transmissive element 37 disperses the UV light such that some or all of the surfaces 36a-36e, or other surfaces of the access port 34, are illuminated by the UV light. In certain embodiments, the illuminator comprises one or more reflective elements 38 positioned to reflect the UV light onto surfaces of the access port 34. In certain embodiments, the transmissive element 37 is configured to illuminate certain surfaces of the access port 34 directly and the reflective elements 38 are configured to reflect light onto other surfaces of access port 34. It will be apparent to one of skill in the art that an illuminator can be configured various combinations of transmissive and reflective elements to direct the collimated light provided by the optical fibers 107 in any desired pattern. In certain embodiments, the illuminator can be configured to expose some surfaces, for example the tip 36a that immediately surrounds the channel 34a that will convey the medical fluid, to a higher intensity of UV light that other surfaces of the access port 34. In certain embodiments, the surfaces include all surfaces of the access port 34 that are wetted by a fluid conveyed through the mating connector (not shown in FIG. 4B) of an IV set or other device, for example a syringe. In certain embodiments, the surfaces of the access port 34 that are exposed are predetermined.

FIG. 4C depicts the internal details of another embodiment of a sterilization cap 104b according to certain aspects of the present disclosure. In this embodiment, the illuminator comprises a source 39 that generates UV light. In certain embodiments, the illuminator of sterilization cap 104b includes reflective elements 38 and is configured to direct the UV light from the UV light source 37b in a manner similar to sterilization cap 104a of FIG. 4B. In certain embodiments, the illuminator comprises transmissive elements, such as transmissive element 37, that redirect the light from the source 39. The cable 106b comprises electrical wires 107b that power the UV light source 39. In certain embodiments, the UV light source 39 comprises a Light Emitting Diode (LED). In certain embodiments, the LED is configured to preferentially provide UV light. In certain embodiments, the LED is configured to preferentially provide UVC light. In certain embodiments, the source 102 provides power of a determined voltage and frequency through the cable 106b to the UV light source 39.

FIG. 4D depicts a sterilization cap 104b being used with an access port 40 that comprises a UV-transmissive material according to certain aspects of the present disclosure. In the example of FIG. 4D, the body 41 of the access port 40 comprises a material that is at least partially transmissive to the wavelengths of the light emitted by the UV light source 39 such that a portion of the UV light emitted by the UV light source 39 passes through the body 41 to reach the internal surfaces of the channel 34a, as shown by the arrows in FIG. 4D, thereby disinfecting the internal surfaces of the access port 40.

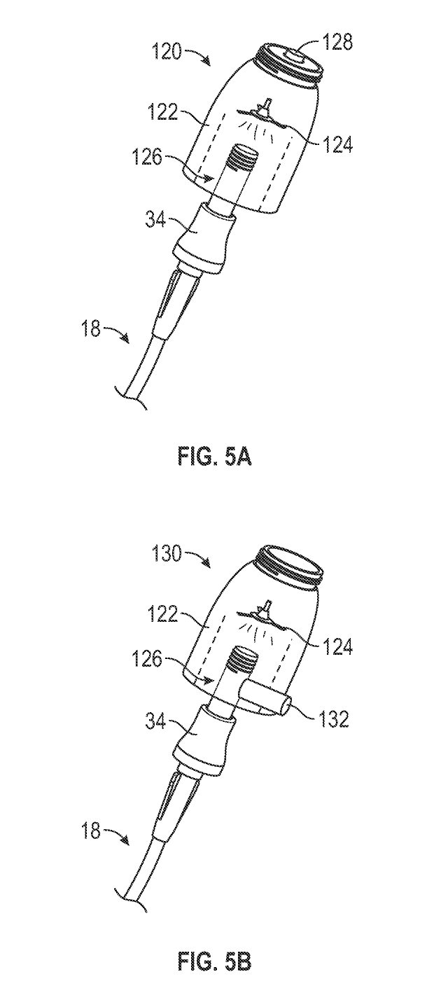

FIGS. 5A and 5B depict additional embodiments of a sterilization system 120 according to certain aspects of the present disclosure. In the example of FIG. 5A, the sterilization system 120 is a portable self-contained device having a body 122 with a UV source 124, for example a UV-emitting LED, located within a cavity 126 that is configured to couple to an access port 34 that is, in this example, part of an IV set 18. The UV source 124 is positioned such that the emitted UV light is directed to the end surface of the access port 34. In certain embodiments, the sterilization system 120 includes an actuator 128, for example a push-button, disposed on an outer surface of the body 122. After the operator couples the sterilization system 120 to the access port, the operator actuates the actuator 128 whereupon the UV light comes on for a predetermined amount of time.

FIG. 5B depicts another embodiment of a sterilization system 130 similar to the sterilization system 120 of FIG. 5A except that the actuator 128 has been replaced by an actuator 132 configured to detect when the sterilization system 130 is fully coupled to the access port 34. In certain embodiments, the sterilization system 130 is configured to automatically actuate the UV source 124 when the actuator 132 detects that the sterilization system 130 is fully coupled to the access port 34. In certain embodiments, the actuator 128 of FIG. 5A is interlocked with the actuator 132 of FIG. 5B such that the actuator 128 does not actuate the UV source 124 unless the actuator 132 detects that the sterilization system 130 is fully coupled to the access port 34.

In certain embodiments, the sterilization system 120 comprises transmissive and/or reflective elements, similar to those shown in FIGS. 4A and 4B, that redirect the UV light from the UV source 124 to one or both of the internal and external surfaces of the access port 34. In certain embodiments, the sterilization system 120 comprises a power source, for example a battery, coupled to the UV source 124 and the actuator 128. In certain embodiments, the sterilization system 120 is disposable.

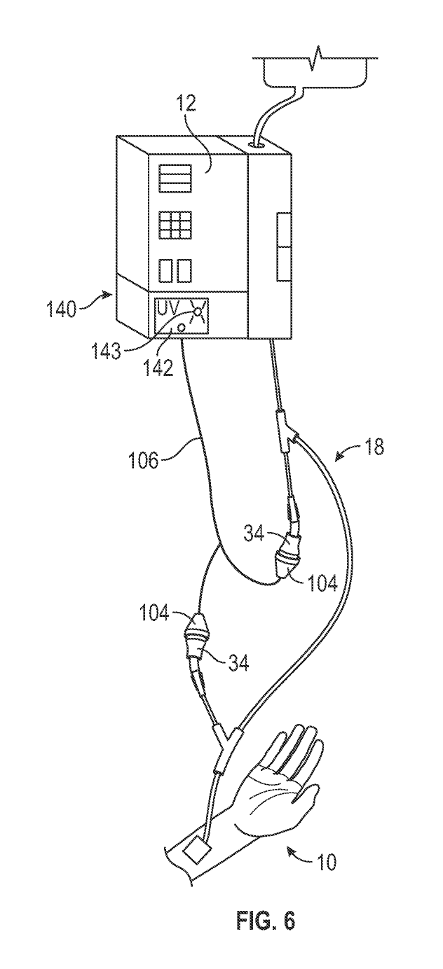

FIG. 6 depicts another embodiment of a sterilization system 140 integrated into an infusion pump 12 according to certain aspects of the present disclosure. The sterilization system 140 comprises a source 142 that is functionally similar to the source 102 of FIG. 4 and configured to be integrated into or mounted compatibly with the infusion pump 12. The embodiment shown in FIG. 6 includes two sterilizing caps 104 connected by a bifurcated cable 106 that, in various embodiments, is an optical cable or electrical cable as discussed with respect to FIG. 4. The plurality of sterilizing caps 104 allows the use of a IV set 18 having access ports 34 at various points that are disinfected, or maintained in a disinfected state, by the plurality of sterilizing caps 104.

In certain embodiments, the sterilization system 140 is functionally connected to the infusion pump 12 such that the infusion pump 12 will not allow the medical fluid to be delivered to the patient unless a signal has been received from the sterilization system 140 indicating that the access port 34 has been disinfected by use of the system 140. In certain embodiments, the system 140 comprises a visual indicator, for example the indicator light 143, that indicates that the system 140 has recently been activated. In certain embodiments, the visual indicator is provided by the sterilizing cap 104 comprising a fluorescent material that glows for a period of time after exposure to the UV light of the illuminator. In certain embodiments, the sterilizing cap 104 comprises a UV-transmissive material such that a portion of the UV light passes through the body of the sterilizing cap 104, for example to energize a fluorescent coating on the outside of the body of the sterilizing cap 104. In certain embodiments, the access port 34 comprises a fluorescent material.

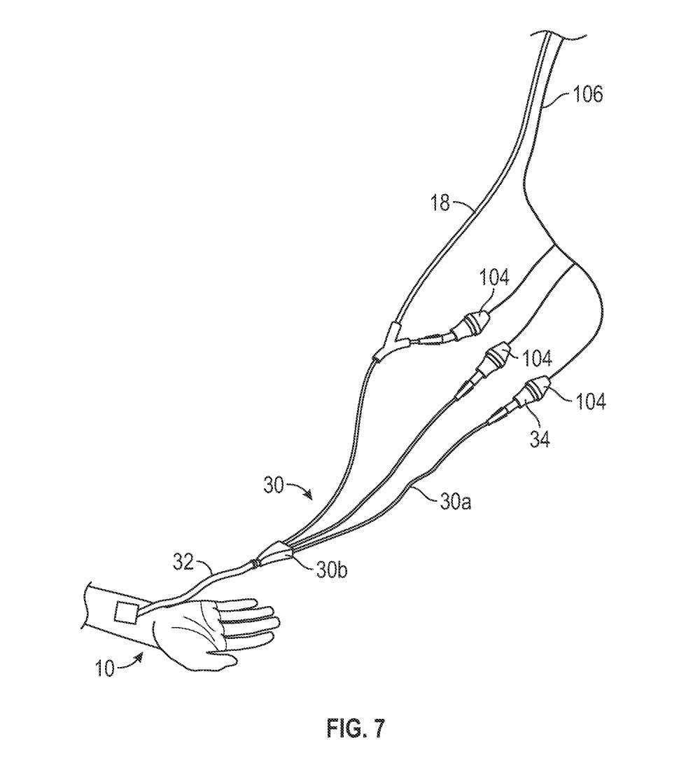

FIG. 7 depicts a detail of multiple sterilizing caps 104 of a sterilizing system coupled to multiple access ports of a CVC 30 according to certain aspects of the present disclosure. A single cable 106 runs alongside the IV line 18 and then divides repeatedly to connect to, in this example, three sterilizing caps 104. The CVC 30 has a venous lumen 32 inserted into a vein of a patient 10 and three access ports 34 individually connected to the lumen 32. In this example embodiment, a medical fluid can be provided continuously through the line 18 to the lumen 32 while the three access ports 34 are maintained in a sterilized condition by the sterilizing caps 104, allowing other medical fluids to be provided through these three access ports 34 without disturbing the existing connection.

In certain embodiments, the CVC 30 may be configured to guide a portion of the UV light received from the sterilization port 104 along at least a portion of the tubes 30a and through fittings 30b, for example by a reflective coating (not shown in FIG. 7) applied to the outside of the tubes 30a and fittings 30b. In certain embodiments, the CVC 30 may be configured to conduct the UV light into the venous lumen 32, thereby disinfecting the portion of the CVC 30 that is located inside the body of patient 10. In certain embodiments, a sterilization cap 104 may be coupled to an access port 34 of the CVC 30 that is located close to the venous lumen 32 so as to increase the amount of UV light reaching the venous lumen 32.

Sterilization systems may be configured, in various embodiments, to provide a short high-intensity flash of UV light that is sufficient to achieve the desired kill rate and/or a continuous low-level exposure to UV light that will achieve the desired kill rate over a longer period of time as well as maintain a sterilized surface in a clean condition. For example, the sterilizing system 100 of FIG. 4A may be configured to continuously provide UV light through the sterilizing cap 104 at a level that is “eye safe” and, when the actuator 108 is actuated, provide a short flash sufficient to sterilize an access port 34.

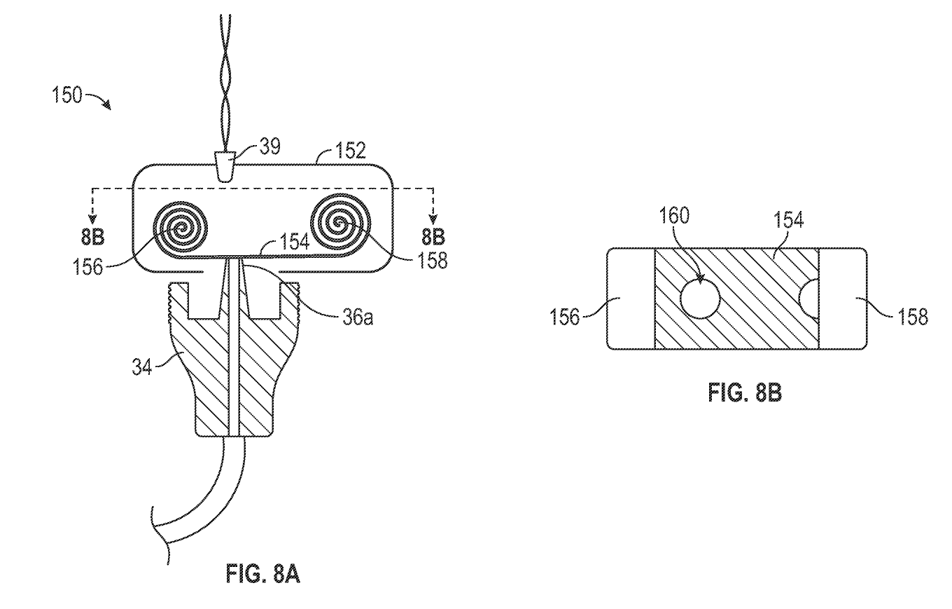

FIGS. 8A and 8B depict a sterilizing system 150 that provides mechanical cleaning and UV sterilization according to certain aspects of the present disclosure. In this embodiment, a housing 152 contains a supply roll 156 of a wiping medium 154 with a take-up roll 158. The system 150 also includes UV source 39. The housing 152 is configured to attach to an access port 34 and, upon actuation, illuminate at least the tip 36A of the access port 34 to UV light then wipe the tip 36a with the wiping medium 154 and advancing the wiping medium 154 onto the take-up roll 158 so that new wiping medium is used for each sterilization operation.

FIG. 8B is a view of internal components of the sterilizing system 150 as indicated by the section line B-B in FIG. 8A. It can be seen that the wiping medium 154 has, in this embodiment, holes 160 configured such that the tip 36a is exposed at certain positions of the wiping medium 154 from roll 156 to roll 158 so as to allow UV light from the source 39 to illuminate the tip 36a. As the wiping medium 154 is advanced, the portions of the wiping medium 154 between the holes will mechanically wipe and clean the tip 36a.

In summary, a sterilizing system that generates and distributes UV light, particularly UVC light, to access ports of IV sets and other points of access to a patient's circulatory system is disclosed. Reduction of many pathogen populations by 99% or more can be routinely accomplished in a timeframe of seconds using UV light intensities that are safe for prolonged exposure. Continuous irradiation may provide kill rates of 99.999% or more. One exemplary application is a CVC having multiple access ports, wherein the CVC remains inserted in a patient's vein for an extended period of time. Use of the UV-based sterilizing procedure, either on a periodic basis immediately prior to use or after disconnection of an IV line from an access port at an intensity sufficient to sterilize the access port within seconds, or on a continual basis at a reduced intensity to maintain the access port in a disinfected condition, reduces the likelihood of a BSI. In some embodiments, the UV device is used to cap the hub and bathe it in UV when it is not connected and in-use. The cap serves to protect the surface and can, in certain embodiments, continuously disinfect it. Stand-alone embodiments can be self-powered and attached to hubs. Tethered devices may utilize a central UV source located proximate to the infusion pump that delivers UV light through one or more optical conduit to multiple hubs. It can be appreciated that access ports are but one example of devices with which the disclosed apparatus and methods may be used, and that this apparatus and method can be extended to a variety of potential infection entry points in a hospital setting involving medical apparatus.

It is understood that the specific order or hierarchy of steps or blocks in the processes disclosed is an illustration of exemplary approaches. Based upon design preferences, it is understood that the specific order or hierarchy of steps or blocks in the processes may be rearranged. The accompanying method claims present elements of the various steps in a sample order, and are not meant to be limited to the specific order or hierarchy presented.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims.

A phrase such as an “aspect” does not imply that such aspect is essential to the subject technology or that such aspect applies to all configurations of the subject technology. A disclosure relating to an aspect may apply to all configurations, or one or more configurations. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. A phrase such an embodiment may refer to one or more embodiments and vice versa.

The word “exemplary” is used herein to mean “serving as an example or illustration.” Any aspect or design described herein as “exemplary” is not necessarily to be construed as preferred or advantageous over other aspects or designs.

Reference to an element in the singular is not intended to mean “one and only one” unless specifically so stated, but rather “one or more.” Unless specifically stated otherwise, the term “some” refers to one or more.

To the extent that the term “include,” “have,” or the like is used in the description or the claims, such term is intended to be inclusive in a manner similar to the term “comprise” as interpreted when employed as a transitional word in a claim.

Pronouns in the masculine (e.g., his) include the feminine and neuter gender (e.g., her and its) and vice versa. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. §112, sixth paragraph, unless the element is expressly recited using the phrase “means for” or, in the case of a method claim, the element is recited using the phrase “operation for.”

Although embodiments of the present disclosure have been described and illustrated in detail, it is to be clearly understood that the same is by way of illustration and example only and is not to be taken by way of limitation, the scope of the present invention being limited only by the terms of the appended claims.

1. An apparatus configured to disinfect an intravenous (IV) access port, the apparatus comprising at least one sterilizing cap comprising:
a body configured to removably couple to the access port; and
an illuminator coupled to the body, the illuminator configured to provide ultraviolet (UV) light;
wherein the body and illuminator are configured to expose at least one surface of the access port to a dose of UV light.

2. The apparatus of claim 1, wherein the dose comprises at least 0.1 milliwatt-second per square centimeter (mW-sec/cm²) of UV light having a wavelength in the range of 100-280 nanometers (nm).

3. The apparatus of claim 2, wherein the wavelength range is 180-280 nm.

4. The apparatus of claim 2, wherein the dose is at least 1 mW-sec/cm².

5. The apparatus of claim 1, wherein:
the apparatus further comprises:
a light source configured to generate the UV light; and
an optical conduit coupled between the light source and the illuminator, the optical conduit configured to guide the UV light from the light source to the illuminator; and
the illuminator is configured to receive the UV light from the optical conduit and direct the received UV light to the at least one surface of the access port.

6. The apparatus of claim 5, wherein the optical conduit comprises a fiber-optic cable.

7. The apparatus of claim 5, wherein the illuminator comprises at least one of a transmissive element and a reflective element.

8. The apparatus of claim 5, further comprising an actuator coupled to the light source, wherein the light source is further configured to start providing the UV light upon actuation of the actuator.

9. The apparatus of claim 8, wherein the light source is further configured to cease providing the UV light after the UV light has been provided for a predetermined amount of time.

10. The apparatus of claim 8, wherein the light source is further configured to provide the UV light at a first intensity for a predetermined amount of time upon actuation of the actuator and then provide the UV light at a second intensity that is less than the first intensity.

11. The apparatus of claim 1, wherein:
the illuminator comprises a source that generates UV light; and
the apparatus further comprises:
a power source configured to provide electrical power to the UV light source; and
an electrical conductor coupled between the power source and the UV light source, the electrical conductor configured to conduct electrical power from the power source to the UV light source.

12. The apparatus of claim 11, wherein the UV light source comprises at least one light-emitting diode (LED).

13. The apparatus of claim 11, further comprising an actuator coupled to the power source, wherein the power source is further configured to start providing the electrical power upon actuation of the actuator.

14. The apparatus of claim 13, wherein the power source is further configured to cease providing the electrical power after the electrical power has been provided for a predetermined amount of time.

15. The apparatus of claim 13, wherein the power source is further configured to provide the electrical power in a first form for a predetermined amount of time after actuation of the actuator and then provide the electrical power in a second form, the first form configured to cause the UV light source to generate the UV light at a first power level and the second form configured to cause the UV light source to generate the UV light at a second power level that is less than the first power level.

16. The apparatus of claim 15, wherein:
the electrical power is provided as one of a pulse-width modulated (PWM) voltage and a pulse-frequency modulated (PFM) voltage; and
the first form delivers more power than the second form.

17. The apparatus of claim 13, wherein:
the sterilizing cap comprises the actuator; and
the actuator is configured to be automatically actuated when the sterilizing cap is coupled to an access port.

18. The apparatus of claim 13, wherein:
the power source comprises the actuator; and
the actuator is configured to be manually actuated by an operator.

19. The apparatus of claim 1, wherein the illuminator comprises:
a source that generates UV light;
a power source coupled to the UV light source; and
an actuator coupled to the power source, wherein the power source is further configured to start providing electrical power to the UV light source upon actuation of the actuator.

20. The apparatus of claim 19, wherein the power source is further configured to cease providing the electrical power after the electrical power has been provided for a predetermined amount of time.

21. The apparatus of claim 19, wherein the actuator is configured to be automatically actuated when the sterilizing cap is coupled to an access port.

22. The apparatus of claim 19, wherein the actuator is configured to be manually actuated by an operator.

23. A method of disinfecting an intravenous (IV) access port, the method comprising the steps of:
coupling a sterilizing cap to the access port, wherein the sterilizing cap is coupled to a source of ultraviolet (UV) light and is configured to expose at least one surface of the access port to a dose of the UV light;
detecting automatically the completion of the coupling of the sterilizing cap to the access port; and
starting the UV light source automatically upon detecting completion of the coupling of the sterilizing cap to the access port.

24. The method of claim 25, wherein the dose comprises at least 0.1 milliwatt-second per square centimeter (mW-sec/cm²) of UV light having a wavelength in the range of 100-280 nanometers (nm).

25. The method of claim 26, wherein the wavelength range is 180-280 nm.

26. The method of claim 26, wherein the dose is at least 1 mW-sec/cm².

27. The method of claim 25, wherein the sterilizing cap comprises the source of the UV light.

28. The method of claim 29, wherein the source of the UV light comprises a light-emitting diode (LED).

29. The method of claim 25, wherein the sterilizing cap is coupled by a fiber-optic cable to the source of the UV light.

30. A system comprising:
an access port configured to be coupled to tubing, the access port comprising:
a body with an external surface, the access port body comprising a UV-transmissive material; and
a cavity within the body, the cavity having an internal surface; and
a sterilization apparatus comprising:
a body configured to removably couple to the access port; and
an illuminator coupled to the sterilization apparatus body, the illuminator configured to provide ultraviolet (UV) light;
wherein the sterilization apparatus body and illuminator are configured to expose one or more of the internal and external surfaces of the access port to a dose of UV light.

31. The system of claim 30, wherein the dose comprises at least 0.1 milliwatt-second per square centimeter (mW-sec/cm²) of UV light having a wavelength in the range of 100-280 nanometers (nm).

32. The system of claim 31, wherein the wavelength range is 180-280 nm.

33. The system of claim 31, wherein the dose is at least 1 mW-sec/cm².
